(19) United States
(12) Patent Application Publication
(10) Pub. No.: US 2018/0021525 A1
(43) Pub. Date: Jan. 25, 2018

(54) Safe Needle Separator for Dental Syringe

(71) Applicants: Sung Youn Kim, Seongnam-si (KR); Byeong Hwal Park, Cheongju-si (KR)

(72) Inventors: Sung Youn Kim, Seongnam-si (KR); Byeong Hwal Park, Cheongju-si (KR)

(21) Appl. No.: 15/644,254

(22) Filed: Jul. 7, 2017

(30) Foreign Application Priority Data
Jul. 21, 2016 (KR) 10-2016-0092645

(51) Int. Cl. A61M 5/32; A61M 5/34

(52) U.S. Cl. CPC A61M 5/3276; A61M 5/347

(57) ABSTRACT

A safe needle separator for a dental syringe includes: a basket having an internal storage space with an open top; a fixing shaft vertically disposed on a bottom inside the basket; and a rotary plate rotatably disposed on an upper end of the fixing shaft and having a holding hole in which a needle head of a dental syringe is inserted and fixed so that when a user holds a dental syringe and turns the rotary plate clockwise with a needle head fixed in the holding hole, the needle head thread-fastened counterclockwise to the dental syringe is separated from the dental syringe and dropped into the storage space.

### CROSS REFERENCE TO RELATED APPLICATION

The present application claims priority to Korean Patent Application No. 10-2016-0092645, filed Jul. 21, 2016, the entire contents of which is incorporated herein for all purposes by this reference.

### BACKGROUND OF THE INVENTION

### Field of the Invention

The present invention relates to a safe needle separator for a dental syringe, the separator allowing easy removal and disposal of medical wastes such as the needle of a medical syringe without infection and safety accidents.

### Description of the Related Art

A syringe is a medical instrument that is used at a hospital to inject a specific liquid drug into the body or take blood from the body. A syringe is composed of a barrel to be filled with liquid drug, a plunger inserted in the barrel to be pushed to discharge the liquid drug forward, and a needle coupled to the front of the barrel to be inserted into the body. Disposable syringes are generally used to prevent secondary infection and they are usually discarded after being used.

Disposal of syringe needles is usually performed by inserting the head of a needle into a basket having a hole for receiving the needle. When the barrel of a syringe is lifted up with a needle inserted in the hole, the needle is separated from the barrel and dropped into the basket. In this process, the medical wastes such as the barrel with the needle removed, a used alcohol swab, and an ample with liquid drug removed are discarded into the basket

Dental syringes should be made to be able to perform quick and accurate medical treatment in the narrow mouth of a patient. Accordingly, the dental syringes that are currently used are made of specific materials in specific shapes unlike normal syringes, so they are expensive.

Dental syringes are made of metal and an ample filled with liquid drug is coupled to the barrel of the dental syringes. Further, a thread for thread-fastening is formed on the outer side of the front end of the barrel and the inner side of the needle head to prevent unexpected separation of the needle. Further, dental syringe needles are formed at both sides of a needle head, in which the front needle is supposed to be inserted into the body and the rear needle is supposed to be inserted into the ample coupled to the barrel, The needle head is thread-fastened to the front end of the barrel by being turned counterclockwise and separated by being turned clockwise (when the needle to be inserted into the body faces down). Further, a circular ring for inserting a finger is disposed at the rear of the plunger coupled to the rear of the barrel.

As described above, needles and a barrel are thread-fastened in dental syringes, so it is impossible to remove the needles with the baskets used to remove the needles of normal syringes. Accordingly, users are required to separate and discard needles from syringes in person. However, the users may get pricked by a used needle and there is a possibility of secondary infection. Further, needles are located at front and rear of the needle head of dental syringes, so the possibility of secondary infection is much higher than normal syringes.

### DOCUMENTS OF RELATED ART

(Patent Document 1) Korean Utility Model Application Publication No. 20-2008-0001377 (2008 May 27)

(Patent Document 2) Korean Utility Model No. 20-0238184 (2001 Jul. 5)

(Patent Document 3) Korean Utility Model Application Publication No. 20-1998-0059228 (1998 Oct. 26)

### SUMMARY OF THE INVENTION

An object of the present invention is to a safe needle separator for a dental syringe, that separator being able to allow easy separation of needles thread-fastened to a dental syringe.

A safe needle separator for a dental syringe according to an embodiment of the present invention includes: a basket having an internal storage space with an open top; a fixing shaft vertically disposed on a bottom inside the basket; and a rotary plate rotatably disposed on an upper end of the fixing shaft and having a holding hole in Which a needle head of a dental syringe is inserted and fixed so that when a user holds a dental syringe and turns the rotary plate clockwise with a needle head fixed in the holding hole, the needle head thread-fastened counterclockwise to the dental syringe is separated from the dental syringe and dropped into the storage space.

According to the safe needle separators for a dental syringe, a holding hole in which the needle head of a dental syringe is inserted is formed at the rotary plate, so when a user holds a dental syringe and turns the rotary plate clockwise like a millstone with the needle head inserted in the holding hole, the needle head thread-fastened counterclockwise to the dental syringe can be easily separated, whereby allowing for safely discarding medical wastes without secondary infection.

### BRIEF DESCRIPTION OF THE DRAWINGS

The above and other objects, features and other advantages of the present invention will be more clearly understood from the following detailed description when taken in conjunction with the accompanying drawings, in which:

FIG. 1 is a perspective view of a safe needle separator for a dental syringe according to a first embodiment of the present invention

FIG. 2 is a partial enlarged view of the safe needle separator for a dental syringe according to the first embodiment of the present invention;

FIG. 3 is another partial enlarged view of the safe needle separator for a dental syringe according to the first embodiment of the present invention;

FIGS. 4A and 4B are other partial enlarged views of the safe needle separator for a dental syringe according to the first embodiment of the present invention;

FIG. 5 is a view showing use of the safe needle separator for a dental syringe according to the first embodiment of the present invention;

FIG. 6 is a perspective view of a safe needle separator for a dental syringe according to a second embodiment of the present invention;

FIG. 7A. is an exploded perspective view of a safe needle separator for a dental syringe to according to a third embodiment of the present invention;

FIG. 7B is a cross-sectional view of the safe needle separator for a dental syringe according to the second embodiment of the present invention;

FIG. 8A is an exploded perspective view of a safe needle separator for a dental syringe according to a fourth embodiment of the present invention;

FIG. 8B is a cross-sectional view of the safe needle separator for a dental syringe according to the fourth embodiment of the present invention;

FIG. 9A is an exploded perspective view of a safe needle separator for a dental syringe according to a fifth embodiment of the present invention; and

FIG. 9B is a cross-sectional view of the safe needle separator for a dental syringe according to the fifth embodiment of the present invention.

### DETAILED DESCRIPTION OF THE INVENTION

The advantages and features of the present invention, and methods of achieving them will be clear by referring to the exemplary embodiments that will be describe hereafter in detail with reference to the accompanying drawings. However, the present invention is not limited to the embodiments described hereafter and may be implemented in various ways, the exemplary embodiments are provided to complete the description of the present invention and let those skilled in the art completely know the scope of the present invention, and the present invention is defined by the claims. Like reference numerals indicate like components throughout the specification.

When it is determined that detailed description of well-known functions or configurations may make the spirit of the present invention unclear in the following description, the detailed description will be omitted. Further, the terminologies to be described below are ones defined in consideration of their function in the present invention and may be changed by the intention of a user or an operator or a custom. Therefore, the definitions should be construed based on the contents throughout the specification.

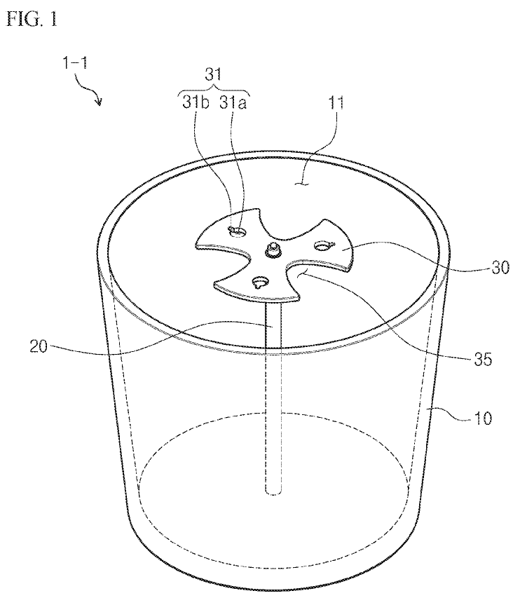

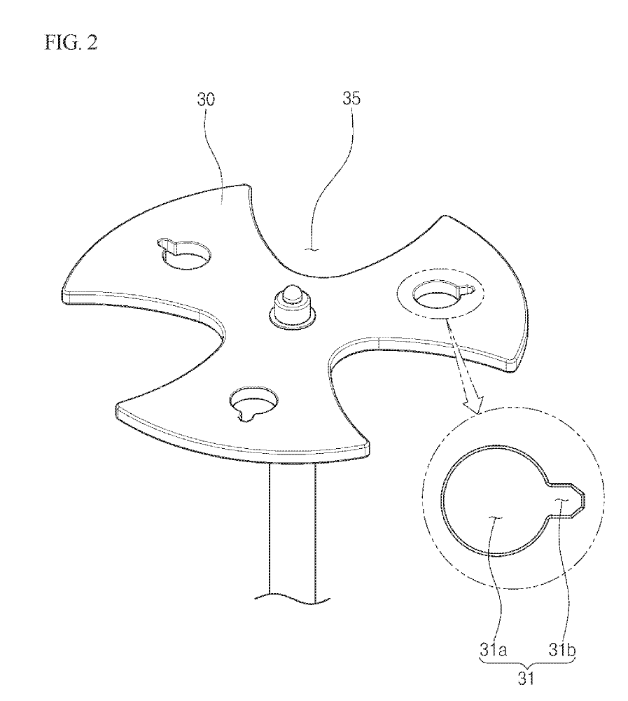

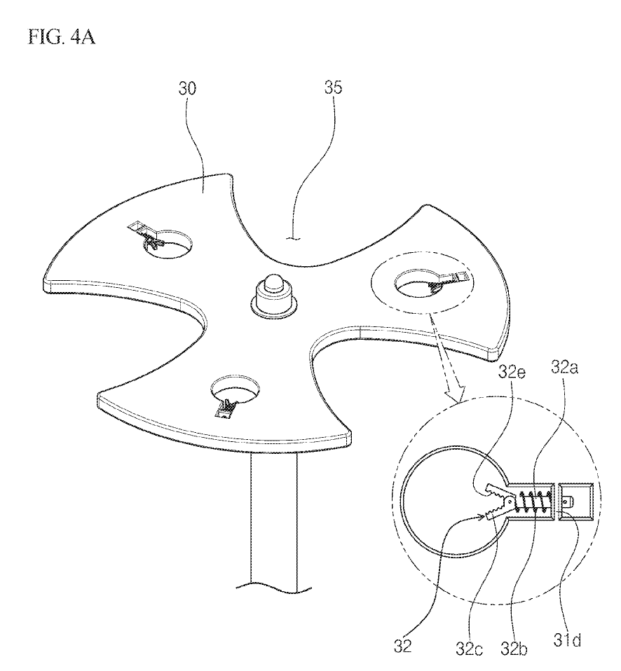

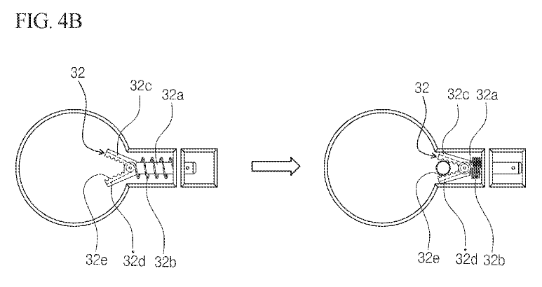

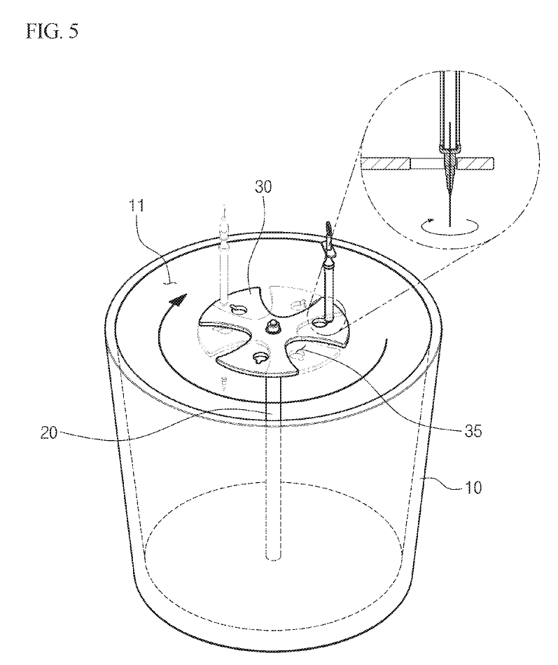

FIGS. 1 to 5 show an embodiment of the present invention. FIG. 1 is a perspective view, FIGS. 2 to 4A are partial enlarged views, FIG. 4B is a view showing operation, and FIG. 5 is a view showing use.

The components and the relationship of the components of a safe needle separator 1-1 for a dental syringe according to a first embodiment of the present invention are described hereafter in detail with reference to FIGS. 1 to 4B.

First, referring to FIG. 1, a basket 10 that is formed to receive and keep needles separated from dental syringes and other various medical wastes has an internal storage space 11 with an open top. The basket 10 may be made of various materials in various sizes and an exclusive basket may be used, but a basket may be reused.

A fixing shaft 20 is vertically erected on the bottom of the basket 10. A rotary plate 30 that will be described below is disposed on the upper end of the fixing shaft 20 to be relatively rotatable. The fixing shaft 20 is formed in a rod shape and firmly fixed to the bottom of the basket 10 not to move when a user 30 rotates the rotary plate 30 with a syringe in his/her hand.

Referring to FIG. 2, the rotary plate 30 is relatively rotatably disposed on the upper end of the fixing shaft 20. When the rotary plate 30 is rotated by a user with a needle head of a dental syringe inserted therein, the needle is separated from the dental syringe. To this end, holding holes 31 in which a needle head of a dental syringe is inserted and fixed are formed through the rotary plate 30.

A plurality of holding holes 30 is formed through the rotary plate 30 and each have an extension 31a inside in which the needle head of a dental syringe is primarily inserted. Further, an insertion 31b that is connected to the extension 31a and is narrower than the extension 31a is formed outside so that the needle head of a dental syringe can be inserted and fixed therein. That is, the holding hole 31 is formed in an 8-shape with the extension 31a positioned at the center and the insertion 31b positional. opposite to the extension 31a in the rotary plate 30. The holding holes 31 may be formed in various sizes so that needle heads of dental syringes can be selectively inserted, depending on the sizes.

The insertions 31b are formed in a polygonal shape so that the needle head of a dental syringe can be fixed without idling therein. Further, throwing grooves 35 are formed by cutting inward the edge of the rotary plate 30 so that medical wastes can be dropped and kept in the storage space.

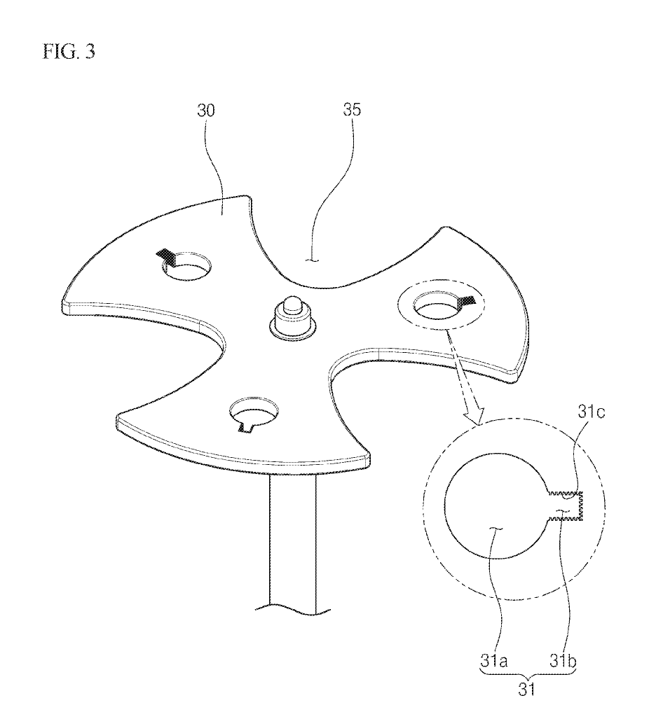

Referring to FIG. 3, a plurality of teeth 31c may be formed inside the insertions 31b so that the needle head of a dental syringe can be fixed without idling.

Referring to FIG. 4A, forceps 32 may be formed in the insertions 31b so that the needle head of a dental syringe can be fixed without idling therein. The forceps 32 are fixed to a mount 31d disposed in the width direction inside the insertion 31b to elastically protrude toward the extension 31a

The forceps 32 includes: a movable shaft 32a that is disposed through the mount 31d to be longitudinally moved in the insertion 31b; a coil spring 32b that is fitted on the front portion of the movable shaft 32a with the rear end supported by the mount 31d to elastically push the movable shaft 32a to the extension 31a; and legs 32c that are formed at the front end of the movable shaft 32a, are supported by the front end of the coil spring 32b, and have a torsion spring 32d therein to move into the insertion 31b together with the needle head of a dental syringe when the movable shaft 32a is pushed into the insertion 31b with the needle head of the dental syringe held between the legs, and to move into the extension 31a from the insertion 31b by the coil spring 32b and drop the needle head by opening by the torsion springs 32d when the pushing force is removed after the needle head is separated from the dental syringe. Teeth 323 may be formed on the inner sides of the legs 32c so that the needle head of a dental syringe can be fixed without idling inside the forceps.

The operation of the forceps 32 is described in detail with reference to FIG. 4B. When the legs 32c elastically pushed to the extension 31a by the coil spring 32b are pushed into the insertion 31b, they are closed by the insertion 31b narrower than the extension 31a. If a needle head is inserted between the legs 32c, it is held by the legs 32c. Further, when the pushing force is to removed, the legs are elastically moved into the extension 31a by the coil spring 32b and are opened by the torsion springs 32d.

The safe needle separator 1-1 for a dental syringe according to the first embodiment of the present invention can be applied to any types of syringes as long as the needle head and the syringe are thread-fastened, such as a leer lock syringe, in addition to a dental syringe.

The method of using the safe needle separator 1-1 for a dental syringe according to the first embodiment of the present invention is described in detail hereafter with reference to FIG. 5.

First, a user puts the needle head of a dental syringe into the extension 31a. Then, the user moves the needle head into the insertion 31b so that the needle head is fixed in the insertion 31b. The insertion 31b is formed in a polygonal shape, so the needle head can be fixed without idling.

The user holds the dental syringe like holding the handle of a millstone and turns the rotary plate 30 clockwise like a millstone. Accordingly, the needle head thread-fastened counterclockwise to the dental syringe is separated from the dental syringe.

Next, the user pushes the needle head in the insertion using a predetermined tool so that the needle head is dropped into the storage space 11 of the basket 10. Further, if the user wants to also discard the dental syringe, he/she can drop the dental syringe into the storage space 11 of the basket 10 through the throwing grooves 35, and other medical wastes such as an alcohol swab and an ample can be discharged into the storage space 11 in the same way.

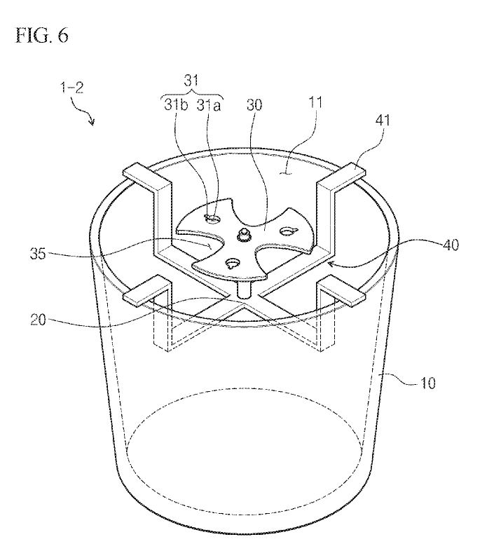

FIG. 6 is a perspective view of a safe needle separator 1-2 for a dental syringe according to a second embodiment of the present invention. The same components as those of the safe needle separator 1-1 for a dental syringe according to the first embodiment of the present invention are given the same reference numerals.

The safe needle separator 1-2 for a dental syringe according to the second embodiment of the present invention according to the second embodiment of the present invention includes: a basket 10 that has an internal storage space 11 with an open top; a holding unit 40 that is disposed at the upper portion in the storage space 11, has legs 41 extending in four directions to be seated on the top of the basket 10, and has a fixing shaft vertically erected at the center of the legs 41; and a rotary plate 30 that is rotatably disposed on the upper end of the fixing shaft and has holding holes 41 in which the needle head of a dental syringe is inserted so that when a user holds a dental syringe and turns the rotary plate 30 clockwise with the needle head fixed in the holding hole 31, the needle head thread-fastened counterclockwise to the dental syringe is separated from the dental syringe and dropped into the storage space 11.

Referring to FIG. 6, the safe needle separator 1-2 for a dental syringe according to the second embodiment of the present invention is similar to or the same as the safe needle separator 1-1 for a dental syringe according to the first embodiment of the present invention in the configuration and operation of the entire components except that it has the holding unit 41 having the legs 41 extending in four directions to be seated on the top of the basket 10 and positioned at the upper portion in the basket 10 and the fixing shaft 42 vertically formed at the center of the legs 41. Accordingly, the configuration and operation can be sufficiently inferred from the first embodiment, so they are not described for simplicity of description.

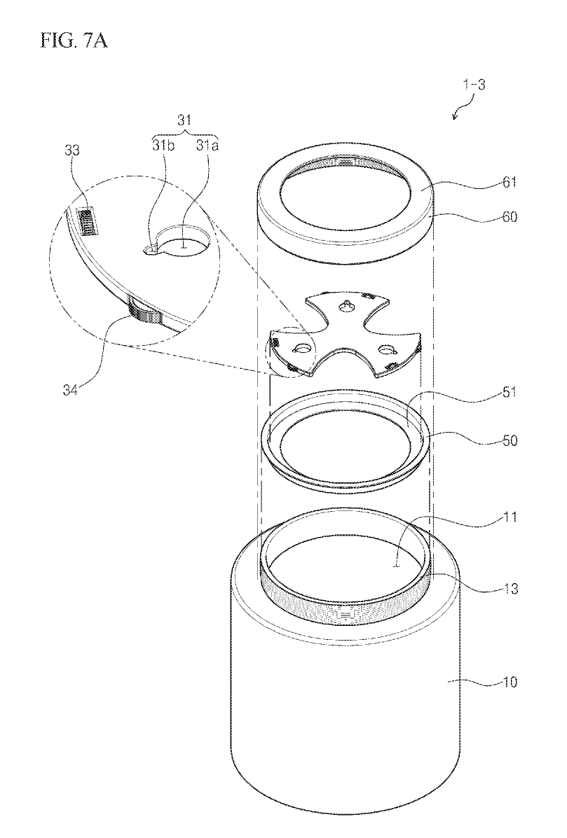

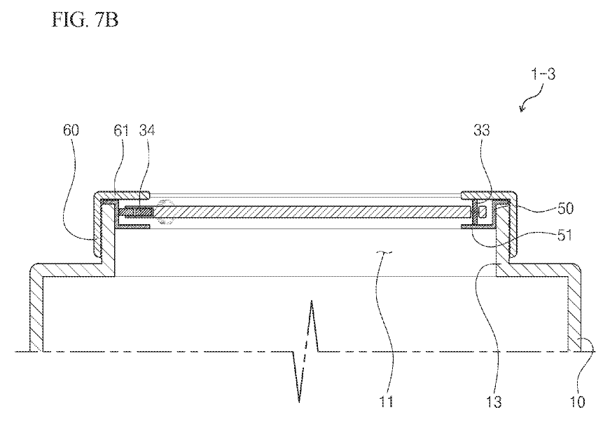

FIGS. 7A and 7B are an exploded perspective view and a cross-sectional view of a safe needle separator 1-3 for a dental syringe according to a third embodiment of the present invention. The same components as those of the safe needle separator 1-1 for a dental syringe according to the first embodiment of the present invention are given the same reference numerals.

The safe needle separator 1-3 for a dental syringe according to a third embodiment of the present invention includes: a basket 10 that has an internal storage space 11 with an open inlet 13 at the top; a first fastening unit 50 that has a hole at the center and a first step 51 on the inner side and is inserted in the inlet 13; a rotary plate 30 that is rotatably seated on the first step 51 and has holding holes 31 in which the needle head of a dental syringe is inserted and fixed; and a cover 60 that is separably coupled to the inlet 13, has a hole at the center, and has an extension 61 protruding toward the center to retain the rotary plate 30 so that when a user holds a dental syringe and turns the rotary plate 30 clockwise with the needle head fixed in the holding hole 31, the needle head thread-fastened counterclockwise to the dental syringe is separated from the dental syringe and dropped into the storage space 11.

The rotary plate 30 has first rollers 33 rolling on the first step 51 and the extension 61 and second rollers 34 rolling on the side of the rotary plate 30 and the inner side of the first fastening unit 50.

Referring to FIGS. 7A and 7B, the safe needle separator 1-3 for a dental syringe according is to the third embodiment of the present invention is similar to or the same as the safe needle separator 1-1 for a dental syringe according to the first embodiment in the configuration and operation of the entire components except that the top of the basket 10 is not fully open but has the protruding inlet 13, the first fastening unit 50 is inserted in the inlet 13, the rotary plate 30 is rotatably seated on the first step 51 of the first fastening unit 50, the rotary plate 30 has the first rollers 33 and the second rollers 34. and the cover 60 having the extension 61 is coupled to the upper end of the inlet 13 in which the first fastening unit 50 and the rotary plate 30 are disposed, so a needle is separated when the rotary plate 30 is rotated between the first fastening unit 50 and the cover. Accordingly, the configuration and operation of the entire components can be sufficiently inferred from the safe needle separator 1-1 for a dental syringe according to the first embodiment, so they are not described for simplicity of description.

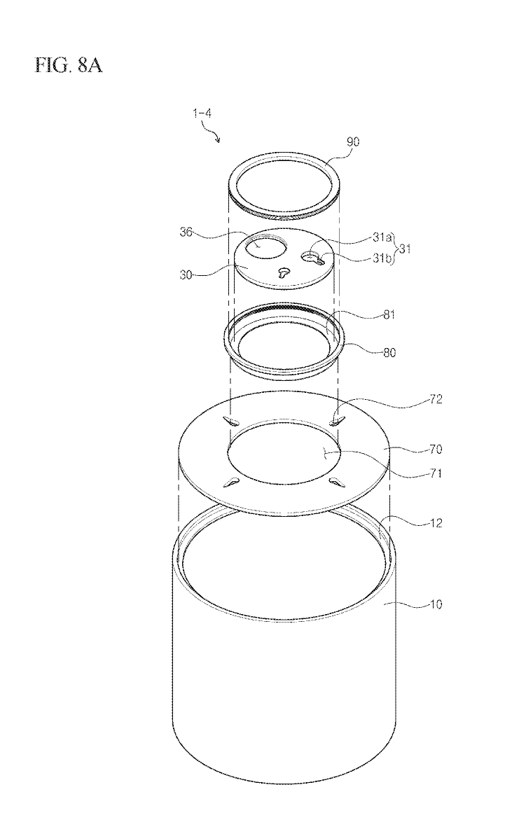

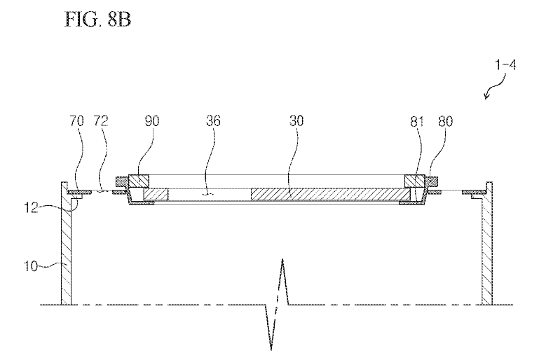

FIGS. 8A and 8B are an exploded perspective view and a cross-sectional view of a safe needle separator 1-4 for a dental syringe according to a fourth embodiment of the present invention. The same components as those of the safe needle separator 1-1 for a dental syringe according to the first embodiment of the present invention are given the same reference numerals.

The safe needle separator 1-4 for a dental syringe according to the fourth embodiment of the present invention includes: a basket 10 that has an internal storage space 11 with an open top and has a second step 12 extending toward the center at the upper end; an intermediate cover 70 that is seated on the second step 12 to cover the upper portion of the basket 10, has a first hole 71 at the center, and has a plurality of insertion holes 72, in which the needle head of a common syringe is inserted to be removed, around the first hole 71 a second fastening unit 80 that is inserted in the first hole 71, has a hole at the center, and has a third step 81 extending toward the center at the lower end; a rotary plate 30 that is rotatably seated on the third step 81 and has holding holes 31 in which the needle head of a dental syringe is inserted and fixed; and an end cover 90 that is separably disposed inside the upper end of the second fastening unit 80 to retain the rotary plate 30 so that when a user holds a dental syringe and turns the rotary plate 30 clockwise with the needle head fixed in the holding hole 31, whereby the needle head thread-fastened counterclockwise to the dental syringe is separated from the dental syringe and dropped into the storage space 11.

The rotary plate 30 has a throwing hole 36 so that a user can drops wastes into the storage space.

Referring to FIGS. 8A and 8B, the safe needle separator 1-4 for a dental syringe according the fourth embodiment of the present invention is similar to or the same as the safe needle separator 1-1 for a dental syringe according to the first embodiment of the present invention except that the second step 12 is formed at the upper end of the basket 10, the intermediate cover 70 having the first hole 71 and the insertion holes 72 for removing the needle of a common syringe is seated on the second step 12, the second fastening unit 80 having the third step 81 for seating the rotary plate 30 is seated in the first hole 71, the end cover 90 is coupled to the second fastening unit 80, and the rotary plate 30 has the throwing hole 36 for dropping wastes, so a needle is separated when the rotary plate 30 is rotated between the second fastening unit 80 and the end cover 90. Accordingly, the configuration and operation of the entire components can be sufficiently referred from the above description and are not described for simplicity of description.

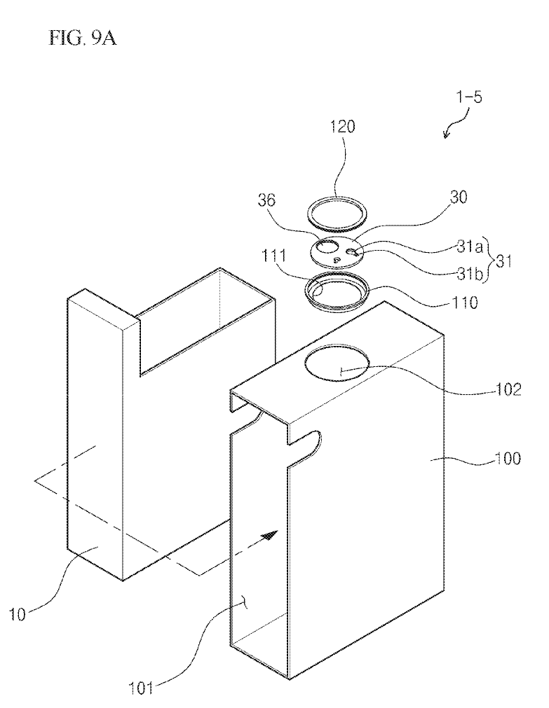

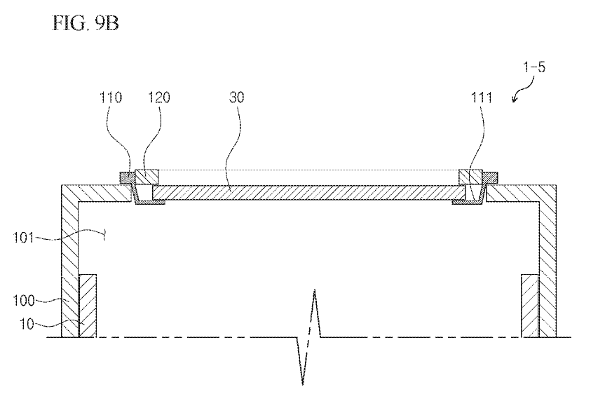

FIGS. 9A and 9B are an exploded perspective view and a cross-sectional view of a safe needle separator 1-5 for a dental syringe according to a fifth embodiment of the present invention. The same components as those of the safe needle separator 1-1 for a dental syringe according to the first embodiment of the present invention are given the same reference numerals.

The safe needle separator 1-5 for a dental syringe according to the fifth embodiment of the present invention includes: a body 100 that has an internal insertion space 101, has a top with a second hole 102, and has an open side; a basket 10 that is disposed in the insertion space 101, slides through the side of the body 100, and has an open top to communicate with the second hole 102; a third fastening unit 110 that is inserted in the second hole 102, has a hole at the center, and has a fourth step 111 extending toward the center at the lower end; a rotary plate 30 that is rotatably seated on the fourth step 111 and has a holding hole 31 in which the needle head of a dental syringe is inserted and fixed; and an end cover 120 that is separably coupled to the upper end of the second fastening unit 80 to retain the rotary plate 30 so that when a user holds a dental syringe and turns the rotary plate 30 clockwise with the needle head fixed in the holding hole 31, the needle head thread-fastened counterclockwise to the dental syringe is separated from the dental syringe and dropped into the storage space 11.

Referring to FIGS. 9A and 9B, the safe needle separator 1-5 for a dental syringe according to the fifth embodiment of the present invention is similar to or the same as the safe needle separator 1-1 for a dental syringe according to the first embodiment and the safe needle separator 1-4 for a dental syringe according to the fourth embodiment in the configuration and operation of the entire components except that the basket 10 slides through the side of the body 100 and the components are installed not at the basket 10, but at the body 100, so that when the rotary plate 30 is rotated between the third fastening unit 110 and the end cover 120, a needle is separated. Accordingly, the configuration and operation of the entire components can be sufficiently inferred from the above description, so they are not described for simplicity of description.

According to the safe needle separators for a dental syringe described above, a holding hole 31 in which the needle head of a dental syringe is inserted is formed at the rotary plate 30, so when a user holds a dental syringe and turns the rotary plate 30 clockwise like a millstone with the needle head inserted in the holding hole 31, the needle head thread-fastened counterclockwise to the dental syringe can he easily separated. Accordingly, it is possible to safely discard medical wastes without secondary infection.

The above description is just an example and the present invention may be easily modified in various ways by those skilled in the art with departing from the scope of the present invention, so the scope of the present invention is not limited to the embodiments.

What is claimed is:

1. A safe needle separator for a dental syringe, the separator comprising:
a basket having an internal storage space with an open top;
a fixing shaft vertically disposed on a bottom inside the basket; and
a rotary plate rotatably disposed on an upper end of the fixing shaft and having a holding hole in which a needle head of a dental syringe is inserted and fixed so that when a user holds a dental syringe and turns the rotary plate clockwise with a needle head fixed in the holding hole, the needle head thread-fastened counterclockwise to the dental syringe is separated from the dental syringe and dropped into the storage space.

2. A safe needle separator for a dental syringe, the separator comprising:
a basket haying an internal storage space with an open top;
a holding unit disposed at an upper portion in the storage space, having legs extending in is four directions to be seated on the top of the basket, and having a fixing shaft vertically disposed at a center of the legs; and
a rotary plate rotatably disposed on an upper end of the fixing shaft and having a holding hole in which a needle head of a dental syringe is inserted so that when a user holds a dental syringe and turns the rotary plate clockwise with a needle head fixed in the holding hole, the needle head thread-fastened counterclockwise to the dental syringe is separated from the dental syringe and dropped into the storage space.

3. A safe needle separator for a dental syringe, the separator comprising:
a basket haying an internal storage space with an open inlet at a top;
a first fastening unit liming a hole at the center and a first step on an inner side and inserted in the inlet;
a rotary plate rotatably seated on the first step and having a holding hole in which a needle head of a dental syringe is inserted and fixed; and
a cover separably coupled to the inlet, having a hole at a center, and having an extension protruding toward a center to retain the rotary plate so that when a user holds a dental syringe and turns the rotary plate clockwise with a needle head fixed in the holding hole_{;} the needle head thread-fastened counterclockwise to the dental syringe is separated from the dental syringe and dropped into the storage space.

4. A safe needle separator for a dental syringe, the separator comprising:
a basket having an internal storage space with an open top and having a second step extending toward a center at an upper end;
an intermediate cover seated on the second step to cover an upper portion of the basket, having a first hole at a center, and having a plurality of insertion holes, in which a needle head of a common syringe is inserted to be removed, around the first hole;
a second fastening unit inserted in the first hole, having a hole at a center, and having a third step extending toward a center at the lower end;
a rotary plate rotatably seated on the third step and having a holding hole in Which a needle head of a dental syringe is inserted and fixed; and
an end cover separably disposed inside an upper end of the second fastening unit to retain the rotary plate so that when a user holds a dental syringe and turns the rotary plate clockwise with a needle head fixed in the holding hole, the needle head thread-fastened counterclockwise to the dental syringe is separated from the dental syringe and dropped into the storage space.

5. A safe needle separator for a dental syringe, the separator comprising:
a body having an internal insertion space, having a top with a second hole, and having an open side;
a basket disposed in the insertion space, sliding through the side of the body, and having an open top to communicate with the second hole;
a third fastening unit inserted in the second hole, having a hole at a center, and having a fourth step extending toward a center at a lower end;
a rotary plate rotatably seated on the fourth step and having a holding hole in Which a needle head of a dental syringe is inserted and fixed; and
an end cover separably coupled to an upper end of the second fastening unit to retain the rotary plate so that when a user holds a dental syringe and turns the rotary plate clockwise with a needle head fixed in the holding hole, the needle head thread-fastened counterclockwise to the dental syringe is separated from the dental syringe and dropped into the storage space.

6. The separator of any one of claim 1, wherein a plurality of holding holes are formed at the rotary plate and each have an extension in which a needle head of a dental syringe is primarily inserted and an insertion connected to the extension and being narrower than the extension so that a needle head of a dental syringe can be inserted and fixed therein.

7. The separator of claim 6, wherein the holding holes are formed in various sizes so that needle heads of dental syringes can be selectively inserted, depending on sizes of the needle heads.

8. The separator of any one of claim 1, wherein the insertions are formed in a polygonal shape so that a needle head of a dental syringe can be fixed therein without idling.

9. The separator of any one of claim 1, wherein teeth are formed inside the insertions so that a needle head of a dental syringe can be fixed in the insertions without

10. The separator of any one of claim 1, wherein forceps are disposed in the insertions to fix a needle head of a dental syringe without idling and are fixed to a mount disposed in a width direction inside insertions to elastically protrude toward the extension.

11. The separator of claim 10, wherein the forceps include:
a movable shaft disposed through the mount to be longitudinally moved in the insertion;
a coil spring fitted on a front portion of the movable shaft with a rear end supported by the mount to elastically push the movable shaft to the extension; and
legs formed at a front end of the movable shaft, supported by a front end of the coil spring, and having a torsion spring therein to move into the insertion together with a needle head of a dental syringe when the movable shall is pushed into the insertion with the needle head of the dental syringe held between the legs, and to move into the extension from the insertion by the coil spring and drop the needle heal by opening by the torsion springs when the pushing force is removed after the needle head is separated from the dental syringe.

12. The separator of claim 11, wherein teeth are formed on inner sides of the legs so that a needle head of a dental syringe can be fixed between the legs without idling.

13. The separator of any one of claim 1, wherein throwing grooves are formed cutting inward an edge of the rotary plate so that wastes can be dropped into the storage space.

14. The separator of claim 3, wherein the rotary plate has first rollers rolling on the first step and the extension and second rollers rolling on a side of the rotary plate and an inner side of the first fastening unit.

15. The separator of claim 4, wherein a throwing hole is formed through the rotary plate so that wastes can be dropped into the storage space.
